(19) United States
(12) Patent Application Publication
(10) Pub. No.: US 2026/0251558 A1
(43) Pub. Date: Aug. 27, 2026

(54) GAS ANALYZER

(71) Applicant: Yokogawa Electric Corporation, Musashino-shi, Tokyo (JP)

(72) Inventors: Arisu Nakajo, Musashino-shi, Tokyo (JP); Hajime Nakamura, Musashino-shi, Tokyo (JP); Yasuhiko Mitsumoto, Musashino-shi, Tokyo (JP)

(73) Assignee: Yokogawa Electric Corporation, Musashino-shi, Tokyo (JP)

(21) Appl. No.: 19/160,139

(22) PCT Filed: Feb. 26, 2024
(86) PCT No.: PCT/JP2024/006861

§ 371 Date: Aug. 27, 2025

(30) Foreign Application Priority Data
Mar. 7, 2023 (JP) 2023-035013

(51) Int. Cl. G01N 21/31; G01N 33/00

(52) U.S. Cl. CPC G01N 21/31; G01N 33/0006; G01N 2201/06113; G01N 2201/0636

(57) ABSTRACT

A gas analyzer includes a light emitter that emits light, an analysis unit that analyzes physical properties of a gas to be measured based on an intensity of the light emitted from the light emitter and transmitted through the gas to be measured, and an adjusting unit including a light transmission space, at least one enclosed container, and a switching unit. The enclosed container, when arranged in the light transmission space and filled with an adjustment gas, allows the light from the light emitter to be transmitted through the adjustment gas and enter the analysis unit, and the switching unit can switch between a first state in which a predetermined enclosed container is arranged in the light transmission space and a second state in which the predetermined enclosed container is not arranged in the light transmission space and the light from the light emitter is allowed to enter the analysis unit.

### CROSS-REFERENCE TO RELATED APPLICATION

The present application claims priority to Japanese Patent Application No. 2023-035013 filed on Mar. 7, 2023, the entire contents of which are incorporated herein by reference.

### TECHNICAL FIELD

The present disclosure relates to a gas analyzer.

### BACKGROUND

A gas analyzer having a light emitter that emits light, an analysis unit that analyzes physical properties of a gas to be measured based on the intensity of the light emitted from the light emitter and transmitted through the gas to be measured, and an adjusting unit that has an adjustment gas reservoir for calibration is known (see, for example, FIG. 7 of Patent Literature (PTL) 1).

### CITATION LIST

### Patent Literature

PTL 1: JP 2019-191154 A

### SUMMARY

### Technical Problem

Gas analyzers such as the one described above preferably have excellent ease, speed, and safety of adjustment, such as calibration and diagnosis.

Therefore, it is an aim of the present disclosure to provide a gas analyzer with excellent ease, speed, and safety of adjustment.

### Solution to Problem

An aspect of the present disclosure is as follows.
[1] A gas analyzer comprising:
a light emitter configured to emit light;
an analysis unit configured to analyze physical properties of a gas to be measured based on an intensity of the light emitted from the light emitter and transmitted through the gas to be measured; and
an adjusting unit including a light transmission space, at least one enclosed container, and a switching unit, wherein
the enclosed container, in a state of being arranged in the light transmission space and filled with an adjustment gas, allows the light emitted from the light emitter to be transmitted through the adjustment gas and enter the analysis unit, and
the switching unit is capable of switching between a first state in which a predetermined enclosed container is arranged in the light transmission space and a second state in which the predetermined enclosed container is not arranged in the light transmission space and the light emitted from the light emitter is allowed to enter the analysis unit.
[2] The gas analyzer according to [1], wherein in the second state, the enclosed container other than the predetermined enclosed container is arranged in the light transmission space.
[3] The gas analyzer according to [1], wherein in the second state, the enclosed container is not arranged in the light transmission space.
[4] The gas analyzer according to [1] or [2], wherein
in the second state, the enclosed container other than the predetermined enclosed container is arranged in the light transmission space, and
the switching unit is capable of switching among the first state, the second state, and a third state in which the enclosed container is not arranged in the light transmission space and the light emitted from the light emitter is allowed to enter the analysis unit.
[5] The gas analyzer according to any one of [1] to [4], wherein
the at least one enclosed container included in the adjusting unit comprises a plurality of enclosed containers, each enclosed container being filled with an adjustment gas having a different concentration or composition, and
the switching unit is capable of selectively arranging each enclosed container among the plurality of enclosed containers in the light transmission space.
[6] The gas analyzer according to [5], wherein the plurality of enclosed containers includes an enclosed container filled with a span calibration gas for a predetermined gas component as the adjustment gas and an enclosed container filled with a zero calibration gas for the predetermined gas component as the adjustment gas.
[7] The gas analyzer according to [5], wherein the plurality of enclosed containers includes an enclosed container filled with a span calibration gas for a predetermined gas component as the adjustment gas, an enclosed container filled with a zero calibration gas for the predetermined gas component as the adjustment gas, and an enclosed container filled with a linearity testing/verification gas for the predetermined gas component as the adjustment gas.
[8] The gas analyzer according to any one of [1] to [7], wherein the at least one enclosed container comprises an enclosed container filled with a light wavelength diagnostic gas component, as the adjustment gas, that serves as a reference for a wavelength of the light.
[9] The gas analyzer according to any one of [1] to [8], wherein
the switching unit includes at least one actuator capable of performing an operation on the light transmission space along with the at least one enclosed container, and the switching unit is capable of switching between the first state and the second state by the operation of the at least one actuator.
[10] The gas analyzer according to [4], wherein
the switching unit includes at least one actuator capable of performing an operation on the light transmission space along with the at least one enclosed container, and
the switching unit is capable of switching among the first state, the second state, and the third state by the operation of the at least one actuator.
[11] The gas analyzer according to any one of [5] to [8], wherein
the switching unit includes at least one actuator capable of performing an operation on the light transmission space along with the at least one enclosed container, and
the switching unit is capable of selectively arranging each enclosed container among the plurality of enclosed containers in the light transmission space by the operation of the at least one actuator.
[12] The gas analyzer according to any one of [9] to [11], wherein the operation of the actuator is rotation.
[13] The gas analyzer according to [12], wherein
the actuator includes a first gear unit,
the switching unit includes a rotary operation unit including a second gear unit configured to engage with the first gear unit,
the actuator is rotated by rotation of the rotary operation unit, and
the adjusting unit includes a cover configured to expose a portion of the rotary operation unit and wholly or partially cover the actuator.
[14] The gas analyzer according to [13], further comprising a drive unit configured to rotationally drive the rotary operation unit.
[15] The gas analyzer according to any one of [9] to [11], wherein the operation of the actuator is sliding.
[16] The gas analyzer according to any one of [8] to [15], wherein
the at least one enclosed container comprises a plurality of enclosed containers arranged side by side in the actuator, and
the operation of the actuator is performed so that the plurality of enclosed containers moves in a direction in which the plurality of enclosed containers is arranged side by side.
[17] The gas analyzer according to [15], wherein the at least one actuator comprises a plurality of actuators capable of performing the operation individually.
[18] The gas analyzer according to any one of [9] to [17], wherein the enclosed container is removably arranged in the actuator.
[19] The gas analyzer according to [18], wherein the actuator includes an actuator body and at least one lid to which the enclosed container can be removably attached, and by the lid being removably attached to the actuator body, the enclosed container removably attached to the lid is stored in an interior space of the actuator body.
[20] The gas analyzer according to any one of [1] to [19], wherein the gas analyzer is an opposing gas analyzer in which the light emitter and the analysis unit are arranged on both sides of the gas to be measured.
[21] The gas analyzer according to any of [1] to [19], wherein the gas analyzer is a reflective gas analyzer comprising a reflector configured to reflect the light emitted from the light emitter and transmitted through the gas to be measured towards the analysis unit, and the light emitter and the analysis unit are arranged on one side of the gas to be measured.
[22] The gas analyzer according to [21], wherein
the adjusting unit includes an adjusting reflector configured to reflect the light and a reflection switching unit capable of switching between a reflective state in which the adjusting reflector is arranged in the light transmission space and a non-reflective state in which the adjusting reflector is not arranged in the light transmission space,
in the reflective state, the adjusting reflector in the first state reflects the light emitted from the light emitter and transmitted through the predetermined enclosed container before the light enters the gas to be measured, and causes the light to be transmitted through the predetermined enclosed container and enter the analysis unit, and
in the non-reflective state, the adjusting reflector in the second state allows the light emitted from the light emitter to be reflected by the reflector and enter the analysis unit.
[23] The gas analyzer according to [22], wherein
the reflection switching unit includes a reflection actuator capable of performing an operation on the light transmission space along with the adjusting reflector, and
the reflection switching unit is capable of arranging the adjusting reflector in the light transmission space by operation of the reflection actuator.
[24] The gas analyzer according to [23], wherein the operation of the reflection actuator is rotation.
[25] The gas analyzer according to [24], wherein
the reflection actuator includes a reflection first gear unit,
the reflection switching unit includes a reflection rotary operation unit including a reflection second gear unit configured to engage with the reflection first gear unit,
the reflection actuator is rotated by rotation of the reflection rotary operation unit, and
the adjusting unit includes a reflection cover configured to expose a portion of the reflection rotary operation unit and wholly or partially cover the reflection actuator.
[26] The gas analyzer according to [25], further comprising a reflection drive unit configured to rotationally drive the reflection rotary operation unit.
[27] The gas analyzer according to [23], wherein the operation of the reflection actuator is sliding.
[28] The gas analyzer according to any one of [22] to [27], wherein the adjusting unit includes a first layer portion that has the enclosed container and the switching unit, a second layer portion that has the adjusting reflector and the reflection switching unit, and the light transmission space that spans the first layer portion and the second layer portion.
[29] The gas analyzer according to any one of [1] to [28], wherein the light emitter is a laser oscillator configured to emit a laser in a predetermined wavelength range as the light.
[30] The gas analyzer according to any one of [1] to [29], wherein the analysis unit performs analysis by absorption spectroscopy.

### Advantageous Effect

According to the present disclosure, a gas analyzer with excellent ease, speed, and safety of adjustment can be provided.

### BRIEF DESCRIPTION OF DRAWINGS

In the accompanying drawings:

FIG. 1A is a front view partially illustrating an adjusting unit in a gas analyzer according to an embodiment;

FIG. 1B is a rear view of the portion illustrated in FIG. 1A;

FIG. 1C is a projection of the portion illustrated in FIG. 1A, viewed at a slant;

FIG. 2 is a conceptual diagram of an opposing gas analyzer;

FIG. 3 is a conceptual diagram of a reflective gas analyzer;

FIG. 4A is a partial cross-sectional projection of an adjusting unit having the portion illustrated in FIG. 1A;

FIG. 4B is a projection of the portion illustrated in FIG. 4A, viewed at a slant;

FIG. 5A is a projection of another example of an adjusting unit, viewed at a slant;

FIG. 5B is a projection partially illustrating the adjusting unit illustrated in FIG. 5A;

FIG. 5C is a projection illustrating the lid of a switching unit in the adjusting unit illustrated in FIG. 5A and an enclosed container attached to the lid;

FIG. 5D is a projection illustrating the lid of the switching unit in the adjusting unit illustrated in FIG. 5A;

FIG. 6 is a schematic diagram illustrating the state of an opposing gas analyzer at the time of adjustment at a measurement point;

FIG. 7 is a schematic diagram illustrating the state of a reflective gas analyzer at the time of adjustment at a measurement point;

FIG. 8 is a schematic diagram illustrating the state of the opposing gas analyzer at the time of adjustment after removal from the measurement point;

FIG. 9 is a front view of another example of the adjusting unit;

FIG. 10 is a front view of another example of the adjusting unit;

FIG. 11A is a front view illustrating a first layer portion in another example of the adjusting unit;

FIG. 11B is a front view of a second layer portion located on the back side of the first layer portion illustrated in FIG. 11A;

FIG. 11C is a schematic diagram illustrating the state of a gas analyzer at the time of adjustment, the gas analyzer having an adjusting unit that has the first layer portion illustrated in FIG. 11A and the second layer portion illustrated in FIG. 11B;

FIG. 11D is a conceptual diagram illustrating the state of the gas analyzer illustrated in FIG. 11C at the time of measurement; and

FIG. 11E is a conceptual diagram illustrating the state of the gas analyzer illustrated in FIG. 11C at the time of adjustment.

### DETAILED DESCRIPTION

Embodiments of the present disclosure are now illustrated in detail with reference to the drawings.

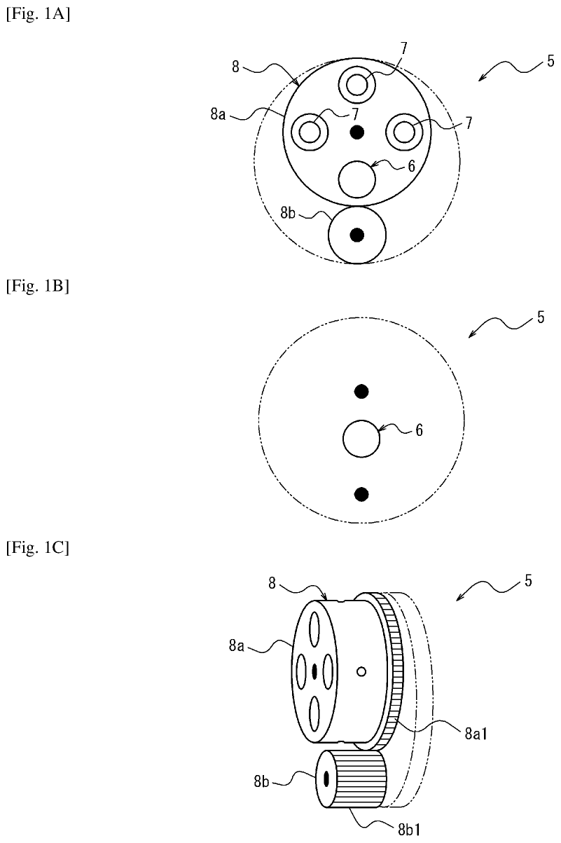

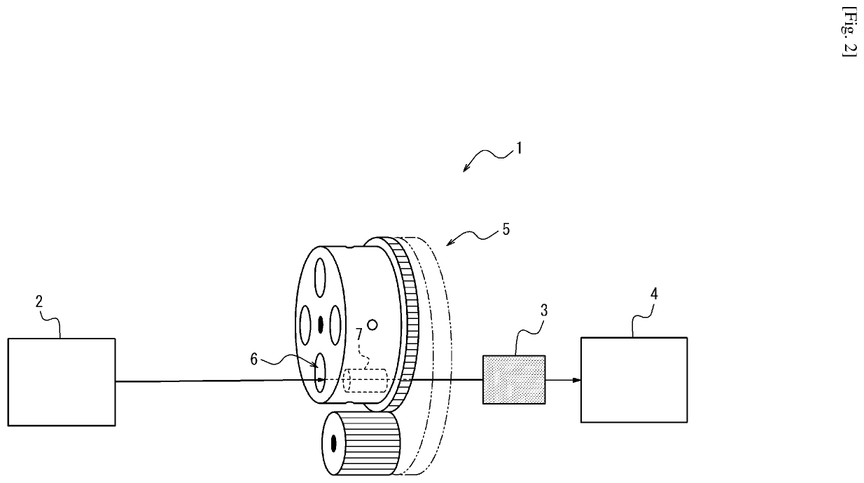

As illustrated in FIGS. 1 to 4B, a gas analyzer 1 according to an embodiment includes a light emitter 2 that emits light, an analysis unit 4 that analyzes physical properties of a gas to be measured 3 based on the intensity of the light emitted from the light emitter 2 and transmitted through the gas to be measured 3, and an adjusting unit 5 including a light transmission space 6, at least one enclosed container 7, and a switching unit 8. The enclosed container 7, in a state of being arranged in the light transmission space 6 and filled with an adjustment gas, allows the light emitted from the light emitter 2 to be transmitted through the adjustment gas and enter the analysis unit 4, and the switching unit 8 is capable of switching between a first state in which a predetermined enclosed container 7 is arranged in the light transmission space 6 and a second state in which the predetermined enclosed container 7 is not arranged in the light transmission space 6 and the light emitted from the light emitter 2 is allowed to enter the analysis unit 4.

According to the above configuration, placement in the first state by the switching unit 8 allows adjustment, such as calibration and diagnosis, using the adjustment gas in the predetermined enclosed container 7, and placement in the second state by the switching unit 8 allows a different adjustment than during the first state, or allows measurement of the gas to be measured 3. The ease, speed, and safety of adjustment can therefore be improved by use of the switching unit 8. Furthermore, according to the above configuration, the ease, speed, and safety of adjustment can also be improved by use of the enclosed container 7 for adjustment. Consequently, according to the above configuration, a gas analyzer 1 with excellent ease, speed, and safety of adjustment can be achieved. The physical properties of the gas to be measured 3 that are analyzed by the analysis unit 4 include, for example, the concentration or presence/ absence of a single or plurality of gas components.

A configuration may be adopted in which in the second state, an enclosed container 7 other than the predetermined enclosed container 7 may be arranged in the light transmission space 6. According to the above configuration, placement in the first state by the switching unit 8 allows adjustment using the adjustment gas in the predetermined enclosed container 7, and placement in the second state by the switching unit 8 allows a different adjustment than during the first state.

A configuration may be adopted in which in the second state, the enclosed container 7 is not arranged in the light transmission space 6. According to the above configuration, placement in the first state by the switching unit 8 allows adjustment using the adjustment gas in the predetermined enclosed container 7, and placement in the second state by the switching unit 8 allows measurement of the gas to be measured 3. In this case, a configuration may be adopted in which nothing is arranged in the light transmission space 6 in the second state, i.e., in which the light transmission space 6 is formed by a cavity in the second state.

A configuration may be adopted in which in the second state, an enclosed container 7 other than the predetermined enclosed container 7 is arranged in the light transmission space 6, and the switching unit 8 is capable of switching among the first state, the second state, and a third state in which the enclosed container 7 is not arranged in the light transmission space 6 and the light emitted from the light emitter 2 is allowed to enter the analysis unit 4. According to the above configuration, placement in the first state by the switching unit 8 allows adjustment using the adjustment gas in the predetermined enclosed container 7, placement in the second state by the switching unit 8 allows a different adjustment than during the first state, and placement in the third state by the switching unit 8 allows measurement of the gas to be measured 3.

The at least one enclosed container 7 included in the adjusting unit 5 includes a plurality of enclosed containers 7, with each enclosed container 7 being filled with an adjustment gas having a different concentration or composition, and the switching unit 8 is capable of selectively arranging each enclosed container 7 among the plurality of enclosed containers 7 in the light transmission space 6. According to the above configuration, a plurality of adjustments can easily be performed by the switching unit 8.

A configuration may be adopted in which the plurality of enclosed containers 7 includes an enclosed container 7 filled with a span calibration gas for a predetermined gas component as the adjustment gas and an enclosed container 7 filled with a zero calibration gas for the predetermined gas component as the adjustment gas. According to the above configuration, span and zero calibration for a predetermined gas component can easily be performed by the switching unit 8. The span calibration gas is a gas containing a predetermined gas component at a predetermined concentration. The zero calibration gas is a gas (such as nitrogen) that substantially does not absorb the light emitted from the light emitter 2.

A configuration may be adopted in which the plurality of enclosed containers 7 includes an enclosed container 7 filled with a span calibration gas for a predetermined gas component as the adjustment gas, an enclosed container 7 filled with a zero calibration gas for the predetermined gas component as the adjustment gas, and an enclosed container 7 filled with a linearity testing/verification gas for the predetermined gas component as the adjustment gas. According to the above configuration, span and zero calibration, along with linearity testing/verification, for a predetermined gas component can easily be performed by the switching unit 8. The linearity testing/ verification gas is a gas containing a predetermined gas component at a concentration of approximately half that of the span calibration gas.

The gas analyzer 1 includes an enclosed container 7 filled with a light wavelength diagnostic gas component, as the adjustment gas, that serves as a reference for the wavelength of the light. According to the above configuration, diagnosis of whether the wavelength of the laser emitted from the light emitter 2 deviates from the reference can easily be performed by the switching unit 8. The peak wavelength of the light wavelength diagnostic gas component is within the wavelength range of the laser and is different from the peak wavelength of a component of the gas to be measured 3 included in the gas to be measured 3. The enclosed container 7 filled with the light wavelength diagnostic gas component may be arranged in the light transmission space 6, and in this state, the gas to be measured 3 may be measured while the wavelength of the laser is adjusted to the reference by the light wavelength diagnostic gas component.

The switching unit 8 includes at least one actuator 8a capable of performing an operation on the light transmission space 6 along with the at least one enclosed container 7, and the switching unit 8 is capable of switching between the first state and the second state by the operation of the at least one actuator 8a. According to the above configuration, the ease of the switching operation by the switching unit 8 can be increased.

The switching unit 8 includes at least one actuator 8a capable of performing an operation on the light transmission space 6 along with the at least one enclosed container 7, and the switching unit 8 is capable of switching among the first state, the second state, and the third state by the operation of the at least one actuator 8a. According to the above configuration, the ease of the switching operation by the switching unit 8 can be increased.

The switching unit 8 includes at least one actuator 8a capable of performing an operation on the light transmission space 6 along with the at least one enclosed container 7, and the switching unit 8 is capable of selectively placing each enclosed container 7 among the plurality of enclosed containers 7 in the light transmission space 6 by the operation of the at least one actuator 8a. According to the above configuration, the ease of the switching operation by the switching unit 8 can be increased.

The operation of the actuator 8a is rotation. According to the above configuration, the ease of the switching operation by the switching unit 8 can be increased.

The actuator 8a includes a first gear unit 8a1, the switching unit 8 includes a rotary operation unit 8b including a second gear unit 8b1 that engages with the first gear unit 8a1, the actuator 8a is rotated by rotation of the rotary operation unit 8b, and the adjusting unit 5 includes a cover 9 that exposes a portion of the rotary operation unit 8b and wholly or partially covers the actuator 8a. According to the above configuration, the cover 9 can prevent foreign matter from entering the light transmission space 6 of the adjusting unit 5, thereby enhancing the accuracy of gas analysis. In addition, by operation of the rotary operation unit 8b by hand, for example, from outside the cover 9, the actuator 8a can be controlled and caused to operate via the rotary operation unit 8b.

A configuration including a drive unit that rotationally drives the rotary operation unit 8b may be adopted. According to the above configuration, the drive unit can cause the actuator 8a to operate via the rotary operation unit 8b. The drive unit is, for example, configured by an actuator such as an electric motor.

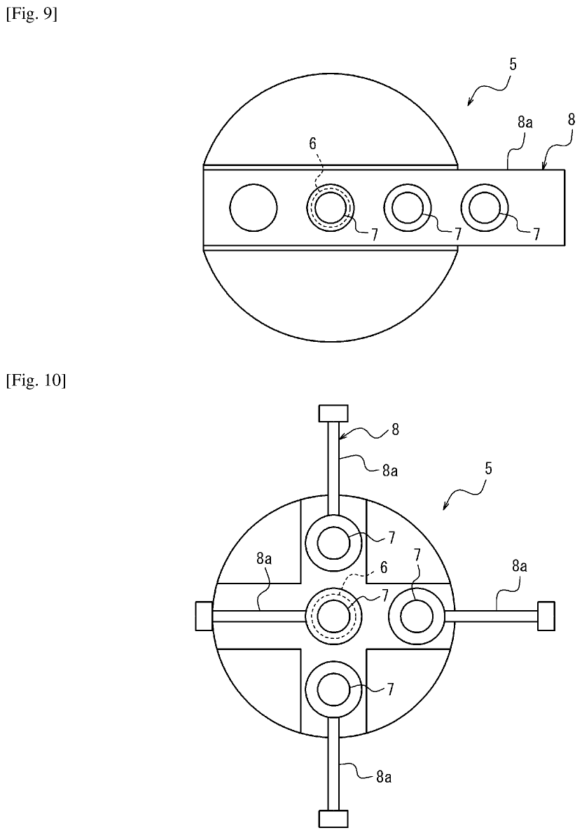

As illustrated in FIG. 9 or FIG. 10, the operation of the actuator 8a may be configured as sliding. According to the above configuration, the ease of the switching operation by the switching unit 8 can be increased.

As illustrated in FIGS. 1A to 1C, the plurality of enclosed containers 7 is arranged side by side in the actuator 8a, and the operation of the actuator 8a is performed so that the plurality of enclosed containers 7 moves in the direction in which the plurality of enclosed containers 7 is arranged side by side. According to the above configuration, the ease of the switching operation by the switching unit 8 can be increased.

As illustrated in FIG. 9, a configuration may be adopted in which the operation of the actuator 8a is sliding, a plurality of the enclosed containers 7 is arranged side by side in the actuator 8a, and the operation of the actuator 8a is performed so that the plurality of enclosed containers 7 moves in the direction in which the plurality of enclosed containers 7 is arranged side by side. According to the above configuration, the ease of the switching operation by the switching unit 8 can be increased.

As illustrated in FIG. 10, a configuration may be adopted in which the operation of the actuator 8a is sliding, and the plurality of actuators 8a is capable of operating individually. According to the above configuration, the ease of the switching operation by the switching unit 8 can be increased.

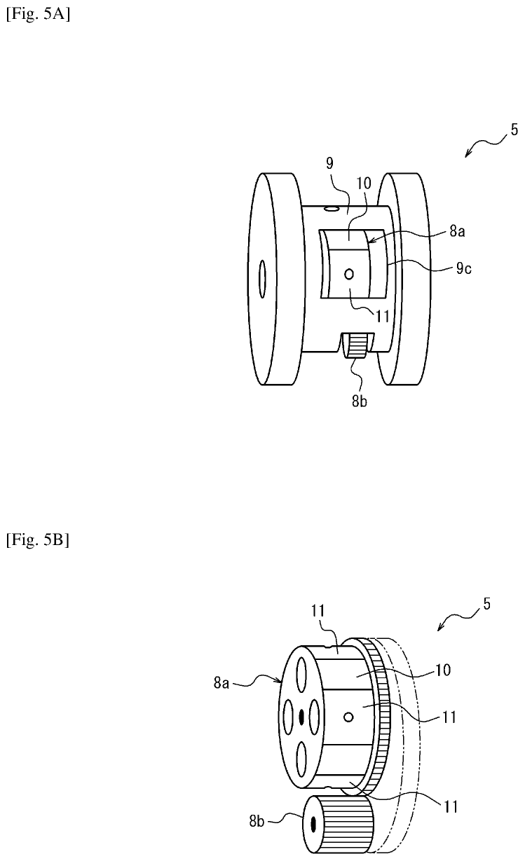

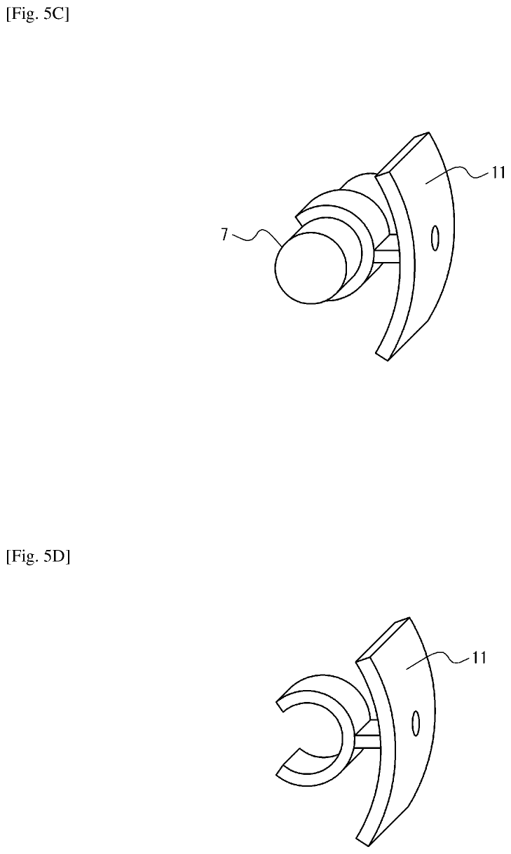

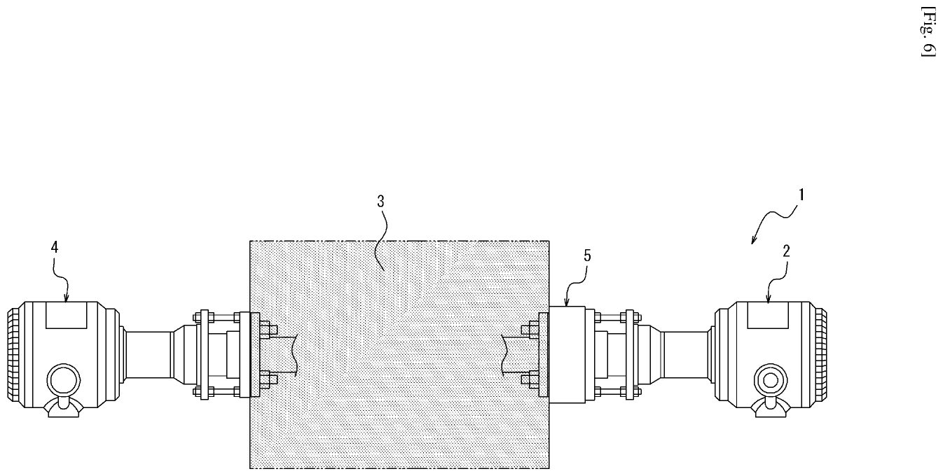

As illustrated in FIGS. 5A to 5D, a configuration may be adopted in which the enclosed container 7 is removably arranged in the actuator 8a. According to the above configuration, a plurality of types of adjustments can be made as needed by attaching and detaching the enclosed container 7.

As illustrated in FIGS. 5A to 5D, a configuration may be adopted in which the actuator 8a includes an actuator body 10 and at least one lid 11 to which the enclosed container 7 can be removably attached, and by the lid 11 being removably attached to the actuator body 10, the enclosed container 7 removably attached to the lid 11 is stored in an interior space of the actuator body 10. According to the above configuration, the ease of attaching and detaching the enclosed container 7 can be increased.

As illustrated in FIG. 5A, the cover 9 may be configured to include a window 9c through which the lid 11 can be passed. According to the above configuration, the ease of attaching and detaching the enclosed container 7 can be increased.

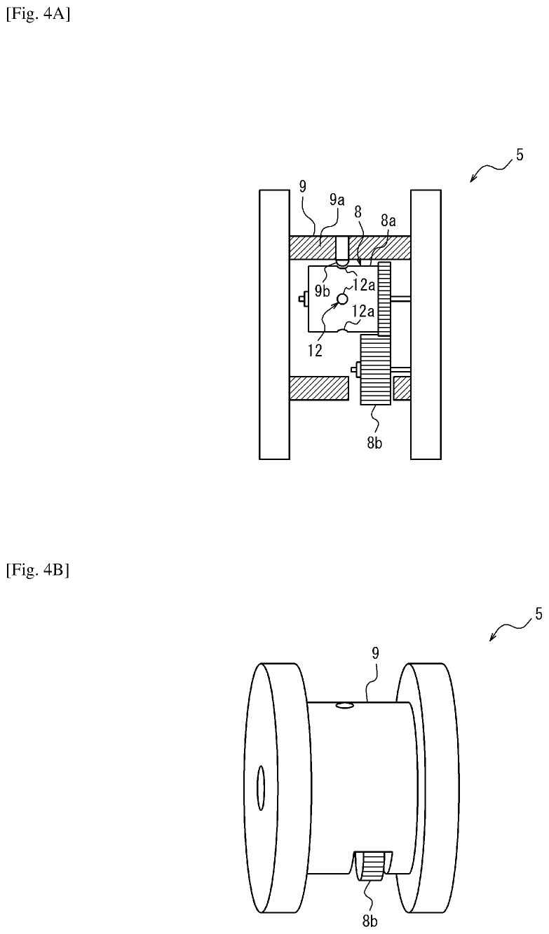

As illustrated in FIG. 4A, the actuator 8a may be configured to include a positioner 12 that hooks against the cover 9 to provide resistance to movement each time switching is performed by the switching unit 8. According to the above configuration, the ease of the switching operation by the switching unit 8 can be increased.

As illustrated in FIG. 4A, the positioner 12 may be configured by a plurality of holes 12a, the cover 9 may include a cover body 9a, a slide member 9b having a convex curved tip surface and slidably arranged in a hole provided in the cover body 9a, and a spring that pushes the slide member 9b towards the inside of the cover 9. Each time the switching is performed by the switching unit 8, the tip surface of the slide member 9b may be configured to selectively enter the plurality of holes 12a acting as the positioner 12. According to the above configuration, the structure of the positioner 12 can be simplified.

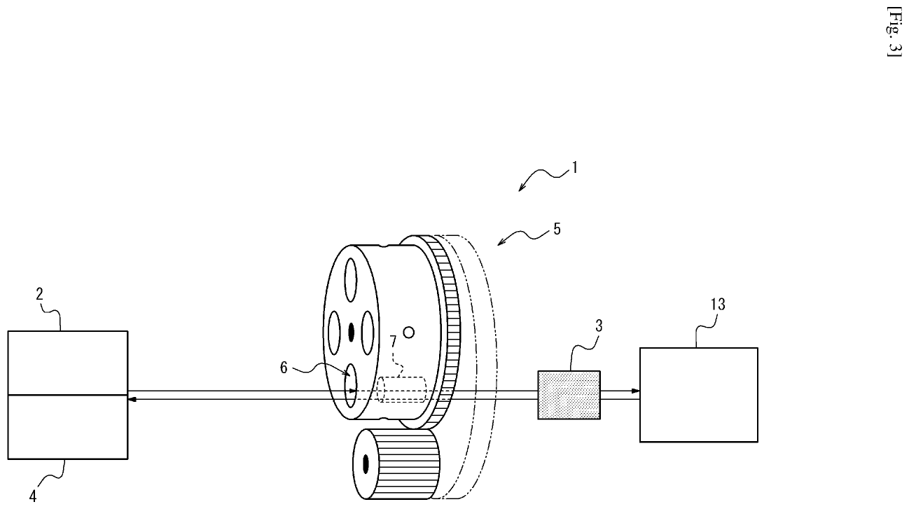

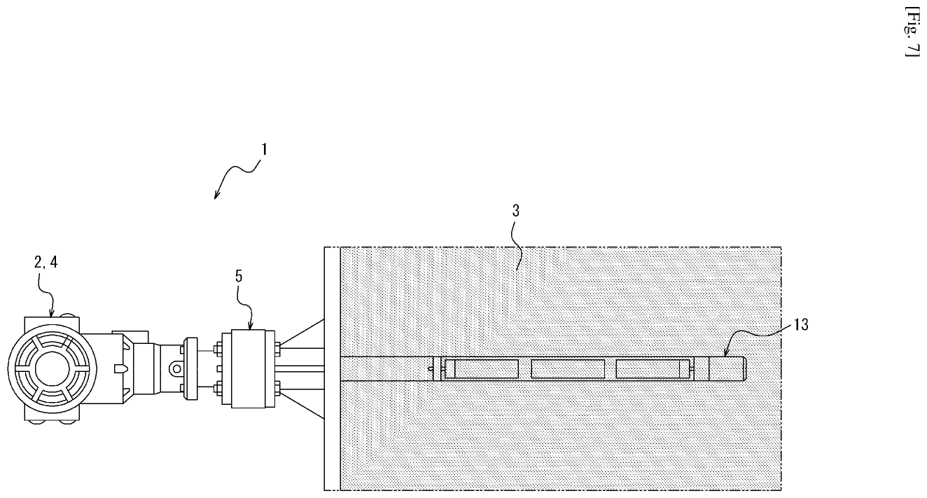

As illustrated in FIG. 2 or FIG. 6, the gas analyzer 1 may be configured as an opposing gas analyzer 1 in which the light emitter 2 and the analysis unit 4 are arranged on opposite sides of the gas to be measured 3. According to the above configuration, the structure of the gas analyzer 1 can be simplified.

In a case in which the gas analyzer 1 is an opposing gas analyzer 1, the adjustment may be performed with the gas analyzer 1 arranged at a location for measuring the gas to be measured 3, as illustrated in FIG. 6, or with the gas analyzer 1 arranged at a location other than the location for measuring the gas to be measured 3, as illustrated in FIG. 8.

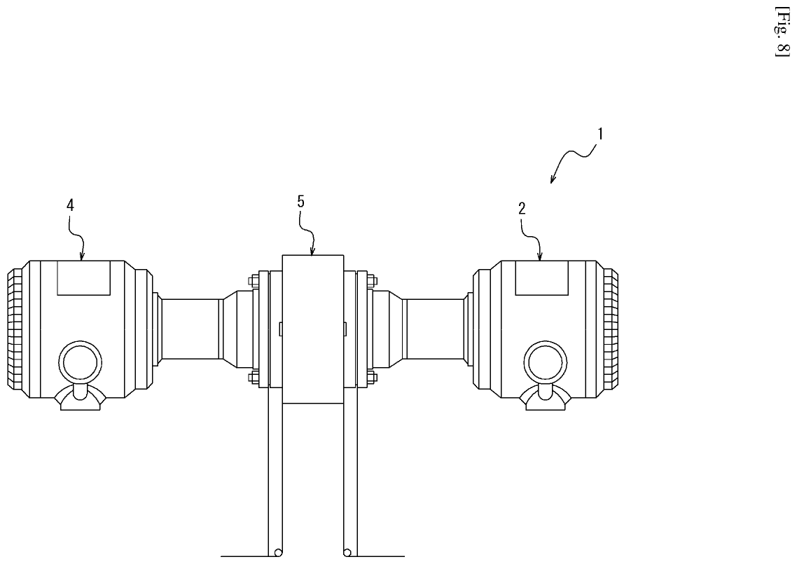

As illustrated in FIG. 3 or FIG. 7, the gas analyzer 1 may be configured as a reflective gas analyzer 1 including a reflector 13 that reflects the light emitted from the light emitter 2 and transmitted through the gas to be measured 3 towards the analysis unit 4, and the light emitter 2 and the analysis unit 4 may be arranged on one side of the gas to be measured 3. According to the above configuration as well, the structure of the gas analyzer 1 can be simplified.

In a case in which the gas analyzer 1 is a reflective gas analyzer 1, the adjustment may be performed with the gas analyzer 1 arranged at a location for measuring the gas to be measured 3, as illustrated in FIG. 7, or with the gas analyzer 1 arranged at a location other than the location for measuring the gas to be measured 3.

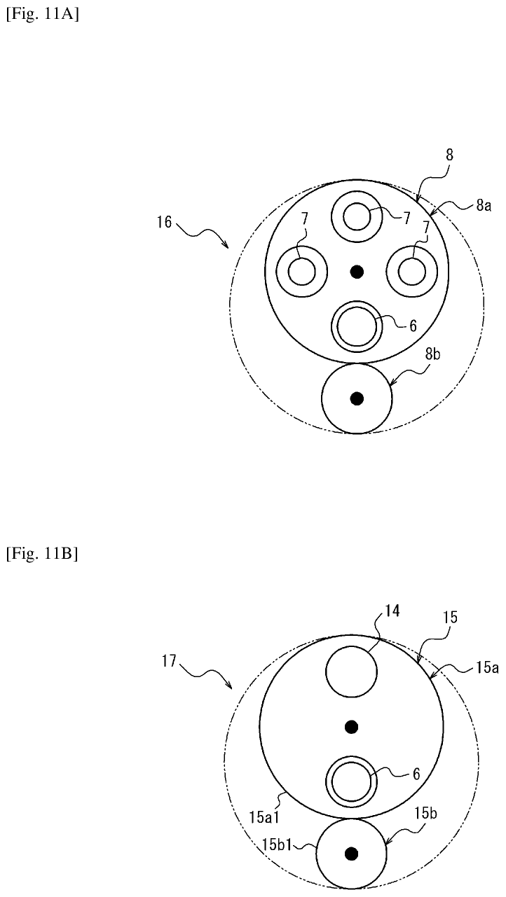

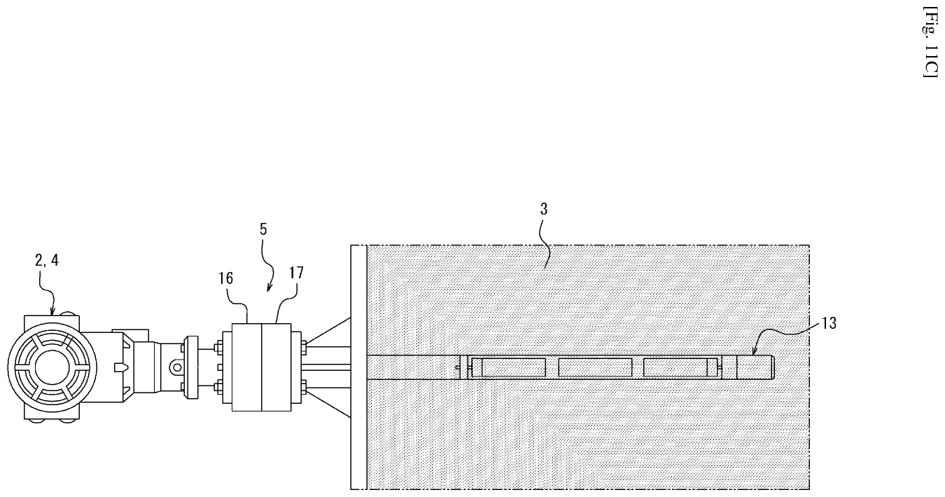

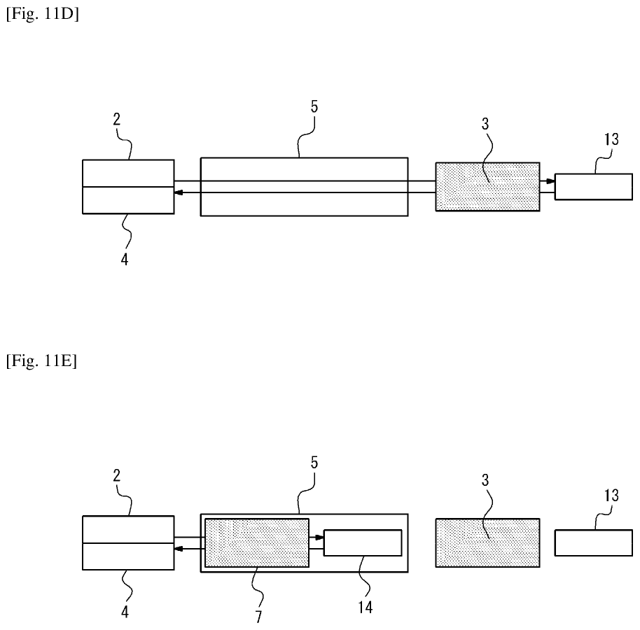

As illustrated in FIGS. 11A to 11E, a configuration may be adopted in which the adjusting unit 5 includes an adjusting reflector 14 that reflects the light and a reflection switching unit 15 capable of switching between a reflective state in which the adjusting reflector 14 is arranged in the light transmission space 6 and a non-reflective state in which the adjusting reflector 14 is not arranged in the light transmission space 6. In the reflective state, the adjusting reflector 14 in the first state reflects the light emitted from the light emitter 2 and transmitted through the predetermined enclosed container 7 before the light enters the gas to be measured 3, and causes the light to be transmitted through a predetermined enclosed container 7 and enter the analysis unit 4. In the non-reflective state, the adjusting reflector 14 in the second state allows the light emitted from the light emitter 2 to be reflected by the reflector 13 and enter the analysis unit 4. According to the above configuration, the influence of the gas to be measured 3 on the adjustment can be suppressed by the reflection switching unit 15 switching to the reflective state, thereby improving the accuracy of the adjustment. The measurement can be performed by the reflection switching unit 15 switching to the non-reflective state. Therefore, according to the above configuration, the accuracy and ease of adjustment by the gas analyzer 1 as a reflective gas analyzer 1 can be improved.

As illustrated in FIG. 11B, a configuration may be adopted in which the reflection switching unit 15 includes a reflection actuator 15a capable of performing an operation on the light transmission space 6 along with the adjusting reflector 14, and the reflection switching unit 15 is capable of arranging the adjusting reflector 14 in the light transmission space 6 by operation of the reflection actuator 15a. According to the above configuration, the ease of the switching operation by the reflection switching unit 15 can be increased.

As illustrated in FIG. 11B, the operation of the reflection actuator 15a may be configured as rotation. According to the above configuration, the ease of the switching operation by the reflection switching unit 15 can be increased.

As illustrated in FIG. 11B, a configuration may be adopted in which the reflection actuator 15a includes a reflection first gear unit 15a1, the reflection switching unit 15 includes a reflection rotary operation unit 15b including a reflection second gear unit 15b1 that engages with the reflection first gear unit 15a1, the reflection actuator 15a is rotated by rotation of the reflection rotary operation unit 15b, and the adjusting unit 5 includes a reflection cover that exposes a portion of the reflection rotary operation unit 15b and wholly or partially covers the reflection actuator 15a. According to the above configuration, the reflection cover can prevent foreign matter from entering the light transmission space 6 of the adjusting unit 5, thereby enhancing the accuracy of gas analysis. In addition, by operation of the reflection rotary operation unit 15b by hand, for example, from outside the reflection cover, the reflection actuator 15a can be controlled and caused to operate via the reflection rotary operation unit 15b.

A configuration including a reflection drive unit that rotationally drives the reflection rotary operation unit 15b may be adopted. According to the above configuration, the reflection drive unit can cause the reflection actuator 15a to operate via the reflection rotary operation unit 15b.

The operation of the reflection actuator 15a may be configured as sliding. According to the above configuration as well, the ease of the switching operation by the reflection switching unit 15 can be increased.

As illustrated in FIGS. 11A to 11C, a configuration may be adopted in which the adjusting unit 5 includes a first layer portion 16 that has the enclosed container 7 and the switching unit 8, a second layer portion 17 that has the adjusting reflector 14 and the reflection switching unit 15, and the light transmission space 6 that spans the first layer portion 16 and the second layer portion 17. According to the above configuration, the structure of the adjusting unit 5 that has the adjusting reflector 14 and the reflection switching unit 15 can be simplified.

The light emitter 2 may be configured as a laser oscillator that emits a laser in a pre-determined wavelength range as the light. According to the above configuration, the accuracy of gas analysis can be increased.

The analysis unit 4 may be configured to perform analysis by absorption spectroscopy. According to the above configuration, the accuracy of gas analysis can be increased.

The gas analyzer 1 may be configured to perform in-situ analysis of a process gas or the like as the gas to be measured 3 in an industrial plant. According to the above configuration, operation of the industrial plant can be made more efficient.

The present disclosure is not limited to the above-described embodiments and can be modified in various ways without departing from the scope thereof.

Therefore, various modifications may be made as long as the gas analyzer 1 includes a light emitter 2 configured to emit light, an analysis unit 3 configured to analyze physical properties of a gas to be measured 3 based on an intensity of the light emitted from the light emitter 2 and transmitted through the gas to be measured 3, and an adjusting unit 5 including a light transmission space 6, at least one enclosed container 7, and a switching unit 8, wherein the enclosed container 7, in a state of being arranged in the light transmission space 6 and filled with an adjustment gas, allows the light emitted from the light emitter 2 to be transmitted through the adjustment gas and enter the analysis unit 4, and the switching unit 8 is capable of switching between a first state in which a predetermined enclosed container 7 is arranged in the light transmission space 6 and a second state in which the predetermined enclosed container 7 is not arranged in the light transmission space 6 and the light emitted from the light emitter 2 is allowed to enter the analysis unit 4.

1. A gas analyzer comprising:
a light emitter configured to emit light;
an analysis unit configured to analyze physical properties of a gas to be measured based on an intensity of the light emitted from the light emitter and transmitted through the gas to be measured; and
an adjusting unit including a light transmission space, at least one enclosed container, and a switching unit, wherein
the enclosed container, in a state of being arranged in the light transmission space and filled with an adjustment gas, allows the light emitted from the light emitter to be transmitted through the adjustment gas and enter the analysis unit, and
the switching unit is capable of switching between a first state in which a predetermined enclosed container is arranged in the light transmission space and a second state in which the predetermined enclosed container is not arranged in the light transmission space and the light emitted from the light emitter is allowed to enter the analysis unit.

2. The gas analyzer according to claim 1, wherein in the second state, the enclosed container other than the predetermined enclosed container is arranged in the light transmission space.

3. The gas analyzer according to claim 1, wherein in the second state, the enclosed container is not arranged in the light transmission space.

4. The gas analyzer according to claim 1, wherein
in the second state, the enclosed container other than the predetermined enclosed container is arranged in the light transmission space, and
the switching unit is capable of switching among the first state, the second state, and a third state in which the enclosed container is not arranged in the light transmission space and the light emitted from the light emitter is allowed to enter the analysis unit.

5. The gas analyzer according to claim 1, wherein
the at least one enclosed container included in the adjusting unit comprises a plurality of enclosed containers, each enclosed container being filled with an adjustment gas having a different concentration or composition, and
the switching unit is capable of selectively arranging each enclosed container among the plurality of enclosed containers in the light transmission space.

6. The gas analyzer according to claim 5, wherein the plurality of enclosed containers includes an enclosed container filled with a span calibration gas for a predetermined gas component as the adjustment gas and an enclosed container filled with a zero calibration gas for the predetermined gas component as the adjustment gas.

7. The gas analyzer according to claim 5, wherein the plurality of enclosed containers includes an enclosed container filled with a span calibration gas for a predetermined gas component as the adjustment gas, an enclosed container filled with a zero calibration gas for the predetermined gas component as the adjustment gas, and an enclosed container filled with a linearity testing/verification gas for the predetermined gas component as the adjustment gas.

8. The gas analyzer according to claim 1, wherein the at least one enclosed container comprises an enclosed container filled with a light wavelength diagnostic gas component, as the adjustment gas, that serves as a reference for a wavelength of the light.

9. The gas analyzer according to claim 1, wherein
the switching unit includes at least one actuator capable of performing an operation on the light transmission space along with the at least one enclosed container, and
the switching unit is capable of switching between the first state and the second state by the operation of the at least one actuator.

10. The gas analyzer according to claim 4, wherein
the switching unit includes at least one actuator capable of performing an operation on the light transmission space along with the at least one enclosed container, and
the switching unit is capable of switching among the first state, the second state, and the third state by the operation of the at least one actuator.

11. The gas analyzer according to claim 5, wherein
the switching unit includes at least one actuator capable of performing an operation on the light transmission space along with the at least one enclosed container, and
the switching unit is capable of selectively arranging each enclosed container among the plurality of enclosed containers in the light transmission space by the operation of the at least one actuator.

12. The gas analyzer according to claim 9, wherein the operation of the actuator is rotation.

13. The gas analyzer according to claim 9, wherein the operation of the actuator is sliding.

14. The gas analyzer according to claim 1, wherein the gas analyzer is a reflective gas analyzer comprising a reflector configured to reflect the light emitted from the light emitter and transmitted through the gas to be measured towards the analysis unit, and the light emitter and the analysis unit are arranged on one side of the gas to be measured.

15. The gas analyzer according to claim 14, wherein
the adjusting unit includes an adjusting reflector configured to reflect the light and a reflection switching unit capable of switching between a reflective state in which the adjusting reflector is arranged in the light transmission space and a non-reflective state in which the adjusting reflector is not arranged in the light transmission space,
in the reflective state, the adjusting reflector in the first state reflects the light emitted from the light emitter and transmitted through the predetermined enclosed container before the light enters the gas to be measured, and causes the light to be transmitted through the predetermined enclosed container and enter the analysis unit, and
in the non-reflective state, the adjusting reflector in the second state allows the light emitted from the light emitter to be reflected by the reflector and enter the analysis unit.

16. The gas analyzer according to claim 15, wherein
the reflection switching unit includes a reflection actuator capable of performing an operation on the light transmission space along with the adjusting reflector, and
the reflection switching unit is capable of arranging the adjusting reflector in the light transmission space by operation of the reflection actuator.

17. The gas analyzer according to claim 16, wherein
the reflection actuator includes a reflection first gear unit,
the reflection switching unit includes a reflection rotary operation unit including a reflection second gear unit configured to engage with the reflection first gear unit,
the reflection actuator is rotated by rotation of the reflection rotary operation unit, and
the adjusting unit includes a reflection cover configured to expose a portion of the reflection rotary operation unit and wholly or partially cover the reflection actuator.

18. The gas analyzer according to claim 15, wherein the adjusting unit includes a first layer portion that has the enclosed container and the switching unit, a second layer portion that has the adjusting reflector and the reflection switching unit, and the light transmission space that spans the first layer portion and the second layer portion.

19. The gas analyzer according to claim 1, wherein the light emitter is a laser oscillator configured to emit a laser in a predetermined wavelength range as the light.

20. The gas analyzer according to claim 1, wherein the analysis unit performs analysis by absorption spectroscopy.
